(19) United States
(12) Patent Application Publication
(10) Pub. No.: US 2023/0364203 A1
(43) Pub. Date: Nov. 16, 2023

(54) New Granules for Animal Feed

(71) Applicant: Novozymes A/S, Bagsvaerd (DK)

(72) Inventors: Flemming Borup, Tygelsjoe (SE); Matthew Kirk Schnorr, Holte (DK); Christian Nyffenegger, Hvidovre (DK); Marianne Thorup Cohn, Nordhavn (DK)

(73) Assignee: Novozymes A/S, Bagsvaerd (DK)

(21) Appl. No.: 18/029,590

(22) PCT Filed: Oct. 7, 2021
(86) PCT No.: PCT/EP2021/077792

§ 371 Date: Mar. 30, 2023

(30) Foreign Application Priority Data
Oct. 7, 2020 (EP) 20200517.9

(51) Int. Cl. A61K 38/44; A23K 20/189; A23K 20/147; A23K 40/30; A23K 20/22; A23K 20/158; A23K 40/10; A23K 20/24; A61K 9/16

(52) U.S. Cl. CPC A61K 38/446; A23K 20/189; A23K 20/147; A23K 40/30; A23K 20/22; A23K 20/158; A23K 40/10; A23K 20/24; A61K 9/1652; A61K 9/1611; A61K 38/44

(57) ABSTRACT

The present invention provides granules for animal feed which comprise a polypeptide having superoxide dismutase activity and a polypeptide having catalase activity and methods for obtaining such.

### REFERENCE TO SEQUENCE LISTING

This application contains a Sequence Listing in computer readable form. The computer readable form is incorporated herein by reference.

### FIELD OF THE INVENTION

The present invention relates to granules for animal feed which comprise a polypeptide having superoxide dismutase activity and a polypeptide having catalase activity and methods for obtaining such.

### BACKGROUND OF THE INVENTION

Animal feed comprising enzymes is known to have several advantages depending on the enzymes used. Typically, the animal feed is found in one of two forms: mash feed composed of all diet components mixed together or pelleted feed where the different diet components are compressed down into pellets with roughly the same size. Pelleted feed is often advantageous for several reasons such as the availability of all needed ingredients and easy storage and handling. Feed pellets may include one or more enzymes and are typically produced by mixing granules comprising the active ingredients such as enzymes with other ingredients such as e.g. cereals and nutrients, followed by conditioning and processing of the mixture into pellets. During the conditioning and pelleting process, the temperature is increased and can in some instances reach high temperatures.

It is important that nutrients and enzymes are evenly distributed in the feed to ensure that all animals receive an optimal blend of nutrients and enzymes via the feed. Furthermore, the high temperatures during the conditioning and pelleting process may negatively affect the stability of the enzyme and thus the activity thereof.

### SUMMARY OF THE INVENTION

The invention provides a granule composition comprising a polypeptide having superoxide dismutase activity and a polypeptide having catalase activity, wherein the molar ratio between the polypeptide having superoxide dismutase activity and the polypeptide having catalase activity is greater than 5:1, such as at least 9:1, such as between 99:1 and 9:1.

In one aspect, the invention provides a composition comprising a polypeptide having superoxide dismutase activity and a polypeptide having catalase activity, wherein upon applying thermal stress to said composition, the activity of the polypeptide having catalase activity in the composition is higher than in a control composition having been applied said thermal stress, wherein said polypeptide having catalase activity is the only polypeptide in said control composition.

In one aspect, the invention provides a co-granulate composition comprising a polypeptide having superoxide dismutase activity and a polypeptide having catalase activity wherein the activity of the polypeptide having catalase activity in the co-granulate composition is higher than in a granulate composition wherein said polypeptide having catalase activity is the only polypeptide, said catalase activity measured upon applying thermal stress to the said co-granulate composition and to said granulate composition.

In one aspect, the invention provides a method of improving the stability of a catalase in a composition comprising adding a polypeptide having superoxide dismutase activity to said composition. Alternatively stated, the invention provides a method of improving the stability of catalase comprising preparing a composition comprising said catalase and a polypeptide having superoxide dismutase activity.

In a further aspect, the invention provides a method of improving the stability of catalase comprising preparing a composition comprising said catalase and a polypeptide having superoxide dismutase activity, wherein upon applying thermal stress to said composition, the activity of the polypeptide having catalase activity in said composition is higher than in a control composition having been applied said thermal stress, wherein said polypeptide having catalase activity is the only polypeptide in said control composition.

In one aspect, the invention provides the use of a polypeptide having superoxide dismutase activity for improving the stability of a catalase in a composition.

In a further use, the invention provides the use of a polypeptide having superoxide dismutase activity in an animal feed additive for improving the stability of a catalase in an animal feed additive.

In one aspect of the invention, the granules comprise a core and a layer surrounding the core. Further provided is a process of preparing the granules of the invention, comprising the steps of:
a. preparing a core comprising a polypeptide having superoxide dismutase activity, a polypeptide having catalase activity and an inert material (e.g. salt, dextrin, cellulose)
b. optionally coating the core with a layer surrounding the core.

The invention also provides an animal feed composition comprising feed components and the granule of the invention, and the use of the granules of the invention for steam treated pelletized feed compositions. The invention further provides a composition wherein the polypeptide having superoxide dismutase activity is selected from the group consisting of SEQ ID NO: 1, SEQ ID NO: 2, SEQ ID NO: 3, SEQ ID NO: 4, and SEQ ID NO: 5, preferably selected from the group consisting of SEQ ID NO: 1, SEQ ID NO: 2 and SEQ ID NO: 5, and polypeptide having catalase activity is selected from the group consisting of SEQ ID NO: 6, SEQ ID NO: 7, SEQ ID NO: 8 and SEQ ID NO: 9, preferably selected from the group consisting of SEQ ID NO: 6 and SEQ ID NO: 7.

### OVERVIEW OF SEQUENCE LISTING

SEQ ID NO 1 is the amino acid sequence of a mature polypeptide having superoxide dismutase (SOD) activity from Trichoderman reesei comprising 287 amino acid residues.

SEQ ID NO 2 is the amino acid sequence of a mature polypeptide having superoxide dismutase (SOD) activity from Aspergillus japonicus comprising 162 amino acid residues.

SEQ ID NO 3 is the amino acid sequence of a mature polypeptide having superoxide dismutase (SOD) activity from Aspergillus templicola comprising 166 amino acid residues.

SEQ ID NO 4 is the amino acid sequence of a mature polypeptide having superoxide dismutase (SOD) activity from Diaporthe nobilis comprising 192 amino acid residues.

SEQ ID NO 5 is the amino acid sequence of a mature polypeptide having superoxide dismutase (SOD) activity from Armillaria ostoyae comprising 159 amino acid residues.

SEQ ID NO 6 is the amino acid sequence of a mature polypeptide having catalase activity from Thermoascus aurantiacus comprising 740 amino acid residues.

SEQ ID NO 7 is the amino acid sequence of a mature polypeptide having catalase activity from Thermoascus aurantiacus comprising 729 amino acid residues.

SEQ ID NO 8 is the amino acid sequence of a mature polypeptide having catalase activity from Thermoascus aurantiacus comprising 729 amino acid residues.

SEQ ID NO 9 is the amino acid sequence of a mature polypeptide having catalase activity from Thermoascus aurantiacus comprising 729 amino acid residues.

SEQ ID NO 10 is the amino acid sequence of a mature polypeptide having catalase activity from Thermoascus aurantiacus comprising 729 amino acid residues.

SEQ ID NO 11 is the amino acid sequence of a mature polypeptide having catalase activity from Thermoascus aurantiacus comprising 729 amino acid residues.

### Definitions

Catalase: A “catalase”, herein also termed “a polypeptide having catalase activity”, may be classified as an EC 1.11.1.6 catalase or as an EP 1.11.1.21 catalase peroxidase.

Composition: The term composition is intended to encompass any granule, granulate, co-granule, co-granulate, solution, liquid, gel, powder, feed additive or feed.

Fungal origin: The term “fungal origin is intended to mean, in reference to a superoxide dismutase or a catalase, that the source of the enzyme is a fungus. A fungus is any member of the group of eukaryotic organisms that includes microorganisms such as yeasts and molds, as well as the more familiar mushrooms. These organisms are classified as a kingdom, fungi. Currently, seven phyla are proposed: Microsporidia, Chytridiomycota, Blastocladiomycota, Neocallimastigomycota, Glomeromycota, Ascomycota, and Basidiomycota. Suitable examples include, without limitation, Trichoderma reesei, Aspergillus versicolor, Aspergillus deflectus, Aspergillus egyptiacus, Westerdykella sp. AS85-2, Aspergillus sp. XZ2669, Preussia terricola, Kionochaeta sp., Metapochonia bulbillosa, Xylomelasma sp. XZ0718, Preussia flanaganii, Cladobotryum sp., Westerdykella sp-46156, Trichoderma hamatum, Mycothermus thermophilus, Cephalotrichiella penicillate, Chaetomium megalocarpum, Chaetomium thermophilum var. thermophilum, Humicola hyalothermophila, Subramaniula anamorphosa, Sphingobacterium sp. T2, Trichoderma rossicum, Trichoderma lixii, Trichoderma sp-54723, Aspergillus niveus, Aspergillus templicola, Pochonia chlamydosporia var. spinulospora, Trichoderma sp-44174, Trichoderma rossicum, Trichoderma sp-54723, Trichoderma sp-44174, Metapochonia suchlasporia, Metarhizium marquandii, Diaporthe nobilis, Tolypocladium sp. XZ2627, Aspergillus japonicus, Metarhizium sp. XZ2431, Armillaria ostoyae, Trichoderma spirale, Aspergillus elegans, Trichoderma sinuosum, Trichoderma virens, Trichoderma hatzianum, Fusicolla acetilerea, Plectosphaerella sp. 1-29, Mariannaea punicea, Penicillium oxalicum, Colletotrichum sp-71086, Aspergillus sp. nov. XZ3202, Trichoderma parapiluliferum, Aspergillus sp. nov. XZ3202, Mucor sp. XZ2651, Rhizomucor miehei, Mucor sp. XZ2651, Amphisphaeriaceae-sp 43674, Humicola fuscoatra and Valsaria rubricosa.

Granule, granulate, co-granule, co-granulate is intended to mean a composition in solid form, such as in layered form or a core comprising the enzymes of the invention and filler allowing for the formation of the solid unit.

Heat Stress: “Heat stress” occurs when an animal's heat load is greater than its capacity to lose heat. Pigs and other animals likely experience headaches, irritability and lethargy when they are too hot and have insufficient water. One or more of the following are typically observed with heat stress: increased breathing rate and sweating, increased water intake, decreased feed intake.

Hydrogenated: The term “hydrogenated” is used for saturation of unsaturated carbohy-drate chains, e.g. in triglycerides, wherein carbon=carbon double bonds are converted to carbon-carbon single bonds.

Mash composition: Mash composition is the nutritionally complete composition of cereals, cereal products and optional supplements in a ground form e.g. comprising wheat, maize, . . . which has not been pelleted and conditioned.

Particle size: By particle size of the granule is meant the mass mean diameter of the granules.

Pelletized feed composition: The term “pelletized feed composition” is intended to mean the feed composition after pelleting and conditioning, i.e. the feed pellets to be fed to the animals.

% RH: The term “% RH” is to be understood as the relative humidity of air. 100% RH is air saturated with water moisture at a fixed temperature and % RH thus reflects the percent moisture saturation of the air.

Solution: A solution is defined as a homogeneous mixture of two or more substances.

Superoxide dismutase: Superoxide dismutase (SOD, EC 1.15.1.1), herein also termed a “polypeptide having superoxide dismutase activity”, is an enzyme that alternately catalyzes the dismutation (or partitioning) of the superoxide (O2-) radical into either ordinary molecular oxygen (O2) or hydrogen peroxide (H2O2).

Suspension: A suspension is defined as fine particles suspended in a fluid.

### DETAILED DESCRIPTION OF THE INVENTION

It has surprisingly found that polypeptides of fungal origin having superoxide dismutase having dismutase activity can formulated so as substantially retain their activity under pelleting conditions; that is stay are substantially stable to thermal stress. However, the same thermal stress conditions associated with pelleting substantially reduce the activity of polypeptides having catalase activity. It has, however, furthermore surprisingly been found that combining a polypeptide having superoxide dismutase activity with a polypeptide having catalase activity provides for a catalase which is substantially stable to the thermal stress associated with pelleting. Accordingly, the polypeptide having superoxide dismutase activity has a stabilizing effect on the activity of the polypeptide having catalase activity.

In one aspect, the invention provides a composition comprising a polypeptide having superoxide dismutase activity and a polypeptide having catalase activity, wherein upon applying thermal stress to said composition, the activity of the polypeptide having catalase activity in the composition is higher than in a control composition having been applied said thermal stress, wherein said polypeptide having catalase activity is the only polypeptide in said control composition.

In one aspect, the invention provides a co-granulate composition comprising a polypeptide having superoxide dismutase activity and a polypeptide having catalase activity wherein the activity of the polypeptide having catalase activity in the co-granulate composition is higher than in a granulate composition wherein said polypeptide having catalase activity is the only polypeptide, said catalase activity measured upon applying thermal stress to the said co-granulate composition and to said granulate composition.

In one aspect, the invention provides the use of a polypeptide having superoxide dismutase activity for improving the stability of a catalase in a composition. Typically, the use is directed to improving the stability to thermal stress, such as when pelleting. Thermal stability, for purposes of comparison, may be performed at temperature of 95° C. and 95% relative humidity, for a conditioning time of 90 sec. These are typical conditions for pelleting such as for the preparation of an animal feed additive.

In one aspect, the invention provides a method of improving the stability of a catalase in a composition comprising adding a polypeptide having superoxide dismutase activity to said composition.

The present invention relates to granules for animal feed which comprise a polypeptide having superoxide dismutase activity and a polypeptide having catalase activity. The inventor of the present invention has with the present invention surprisingly found a method of providing a polypeptide having superoxide dismutase activity and a polypeptide having catalase activity in optimal amounts where even distribution of the enzymes is ensured when provided to animals. In one aspect of the invention, a granule comprising a polypeptide having superoxide dismutase activity and a polypeptide having catalase activity is provided wherein the molar ratio between the polypeptide having superoxide dismutase activity and the polypeptide having catalase activity is greater than 5:1, such as at least 9:1, such as between 99:1 and 9:1.

As stated, the composition of the invention is typically such that the activity of both the catalase and the superoxide dismutase is substantially maintained upon applying thermal stress of the composition. Suitably, at least 60% of the activity of the polypeptide having superoxide dismutase (SOD) activity present in the core of the granules after steam pelleting at 90 degrees Celsius is retained compared to the activity of the polypeptide having superoxide dismutase (SOD) activity in the core of the granules before steam pelleting.

An aspect of the invention is directed to a composition comprising a polypeptide having superoxide dismutase (SOD) activity and polypeptide having catalase activity, wherein the activity of the catalase present in the core of the granules after steam pelleting at 90 degrees Celsius is substantially retained compared to the activity of the catalase in the core of the granules before steam pelleting. Suitably, at least 60% of the activity of the polypeptide having catalase activity present in the core of the granules after steam pelleting at 90 degrees Celsius is retained compared to the activity of the polypeptide having catalase activity in the core of the granules before steam pelleting.

Preferably, retained activity of the polypeptide having catalase activity is at least 65% such as at least 70% such as at least 75%, such as at least 80%, upon applying thermal stress.

Preferably, retained activity of the polypeptide having superoxide dismutase activity is at least 65% such as at least 70% such as at least 75%, such as at least 80%, upon applying thermal stress.

The composition is typically selected from the group consisting of a pellet, such as a feed pellet, a granule, a granulate, an animal feed additive, and an animal feed.

An aspect of the invention is directed to the composition for use as animal feed additive or for use in an animal feed additive. A related aspect is directed to the use of the composition of the invention as an animal feed additive or in the preparation of an animal feed additive.

A further aspect of the invention is directed to a co-granulate composition comprising a polypeptide having superoxide dismutase activity and a polypeptide having catalase activity wherein the activity of the polypeptide having catalase activity in the co-granulate composition is higher than in a granulate composition wherein said polypeptide having catalase activity is the only polypeptide, said catalase activity measured upon applying thermal stress to the said co-granulate composition and to said granulate composition.

In one aspect, the invention provides a method of improving the stability of a catalase in a composition comprising adding a polypeptide having superoxide dismutase activity to said composition. In a further aspect, the invention provides a method of improving the stability of catalase comprising preparing a composition comprising said catalase and a polypeptide having superoxide dismutase activity, wherein upon applying thermal stress to said composition, the activity of the polypeptide having catalase activity in said composition is higher than in a control composition having been applied said thermal stress, wherein said polypeptide having catalase activity is the only polypeptide in said control composition.

In one aspect, the invention provides a method of improving the stability of a superoxide dismutase in a composition comprising adding a polypeptide having catalase activity to said composition. In a further aspect, the invention provides a method of improving the stability of superoxide dismutase comprising preparing a composition comprising said superoxide dismutase and a polypeptide having catalase activity, wherein upon applying thermal stress to said composition, the activity of the polypeptide having superoxide dismutase activity in said composition is higher than in a control composition having been applied said thermal stress, wherein said polypeptide having superoxide dismutase activity is the only polypeptide in said control composition.

For purposes of measuring the retained activity upon application of thermal stress, the thermal stress is suitably steam pelleting at 90 degrees Celsius and 95% relative humidity, for a conditioning time of 90 sec.

A further advantage of the granules of the present invention and the method of producing these is that granules and animal feed produced from the granules are storage and heat stable. Furthermore, the granules and feed pellets from the granules provide a cost efficient and environmentally friendly means for feeding animals optimized feed.

The Granule

When referring to the granule of the present invention it can either be a single granule or several granules.

The granule of the present invention is particularly well suited for steam pelleting and as part of a steam treated pelletized feed composition. The granule comprises a core and a layer surrounding the core, wherein the core comprises a polypeptide having superoxide dismutase activity and a polypeptide having catalase activity.

Suitable particle sizes of the granule of the present invention is found to be 50 μm-2000 μm, more particularly 100 μm-1500 μm. In an embodiment of the invention, the particle size of the granule is more than 250 μm. In a further embodiment of the invention, the particle size is below 1200 μm. In yet a further embodiment, the particle size is between 250-1200 μm. In another embodiment of the present invention the particle size of the finished granule is 250-900 μm. In yet another embodiment of the present invention the mean particle size of the finished granule is 500-700 μm. In still another embodiment of the present invention the particle size of the finished granule is 600-1200 μm. In still another embodiment of the present invention, the particle size of the finished granule is 600-900 μm.

A raw granulate is one which does not comprise a protective coating or outer layer. For example, a raw granulate may be a core than is absent of salt outer layer or coating, or oil or fat or wax outer layer of coating, wherein said outer layer or coating provides increased thermal stability.

The Core

The core comprises a polypeptide having superoxide dismutase activity and a polypeptide having catalase activity.

The core can either be
a homogeneous blend of enzymes including one or more polypeptides having superoxide dismutase activity and one or more polypeptides having catalase activity,

or
an inert particle with polypeptide having superoxide dismutase activity and a polypeptide having catalase activity and optionally further enzymes applied onto it,

or
a homogenous blend of enzymes including one or more polypeptides having superoxide dismutase activity and one or more polypeptides having catalase activity and materials which act as binders which are coated with the one or more polypeptides having superoxide dismutase activity and polypeptides having catalase activity.

The enzymes may be applied to the core in the form of liquid and/or concentrated dry matter. In one aspect, the polypeptide having superoxide dismutase activity is applied to the core in the form of a liquid, in another aspect the polypeptide having superoxide dismutase activity is applied to the core in the form of concentrated dry matter, in yet another aspect the polypeptide having superoxide dismutase activity is applied to the core partly in the form of a liquid and partly in the form of concentrated dry matter. In one aspect, the polypeptide having catalase activity is applied to the core in the form of a liquid, in another aspect the polypeptide having catalase activity is applied to the core in the form of concentrated dry matter, in yet another aspect the polypeptide having catalase activity is applied to the core partly in the form of a liquid and partly in the form of concentrated dry matter.

In the instances where the core comprises an inert particle, the inert particle may be water soluble or water insoluble, e.g. starch, e.g. in the form of cassava or wheat; or a sugar (such as sucrose or lactose), or a salt (such as sodium chloride or sodium sulfate). Suitable inert particle materials of the present invention include inorganic salts, sugars, sugar alcohols, small organic molecules such as organic acids or salts, minerals such as clays or silicates or a combination of two or more of these. Inert particles may be produced by a variety of granulation techniques including crystallization, precipitation, pan-coating, fluid bed coating, fluid bed agglomeration, rotary atomization, extrusion, prilling, spherization, size reduction methods, drum granulation, and/or high shear granulation.

In the instances where the core comprises one or more binders, the binders may be synthetic polymers such as e.g. a vinyl polymer, polyvinyl pyrrolidone (PVP), polyvinyl alcohol (PVA), polyvinyl acetate, polyacrylate, polymethacrylate, polyacrylamide, polysulfonate, polycarboxylate, and copolymers thereof, waxes including fats, fermentation broth, carbohydrates, salts or polypeptides. In a particular embodiment, the binder is a polypeptide.

The polypeptide may be selected from gelatin, collagen, casein, chitosan, poly aspartic acid and poly glutamatic acid. In another particular embodiment the binder is a cellulose derivative such as hydroxypropyl cellulose, methyl cellulose or CMC. A suitable binder is a carbohydrate binder such as dextrin e.g Glucidex 21D or Avedex W80.

In one embodiment, the core may comprise a salt. The salt may be an inorganic salt, e.g. a salt of sulfate, sulfite, phosphate, phosphonate, nitrate, chloride or carbonate or salts of simple organic acids (less than 10 carbon atoms e.g. 6 or less carbon atoms) such as citrate, malonate or acetate. Examples of cations in these salts are alkali or earth alkali metal ions, although the ammonium ion or metal ions of the first transition series, such as sodium, potassium, magnesium, calcium, zinc or aluminium. Examples of anions include chloride, iodide, sulfate, sulfite, bisulfite, thiosulfate, phosphate, monobasic phosphate, dibasic phosphate, hypophosphite, dihydrogen pyrophosphate, carbonate, bicarbonate, metasilicate, citrate, malate, maleate, malonate, succinate, lactate, formate, acetate, butyrate, propionate, benzoate, tartrate, ascorbate or gluconate. In particular alkali- or earth alkali metal salts of sulfate, sulfite, phosphate, phosphonate, nitrate, chloride or carbonate or salts of simple organic acids such as citrate, malonate or acetate may be used. Specific examples include NaH₂PO₄, Na₂HPO₄, Na₃PO₄, (NH₄)H₂PO₄, K₂HPO₄, KH₂PO₄, Na₂SO₄, K₂SO₄, KHSO₄, ZnSO₄, MgSO₄, CuSO₄, Mg(NO₃)₂, (NH₄)₂SO₄, sodium borate, magnesium acetate and sodium citrate. The salt in the core of the particle may also be a hydrated salt, i.e. a crystalline salt hydrate with bound water(s) of crys-tallization, such as described in WO 99/32595. Examples of hydrated salts include magnesium sulfate heptahydrate (MgSO₄(7H₂O)), zinc sulfate heptahydrate (ZnSO₄(7H₂O)), sodium phosphate dibasic heptahydrate (Na₂HPO₄(7H₂O)), magnesium nitrate hexahydrate (Mg(NO₃)₂(6H₂O)), sodium borate decahydrate, sodium citrate dihydrate and magnesium acetate tetrahydrate.

In one embodiment, the core and/or the inner salt layer may comprise a moisture absorbing compound. The moisture absorbing compound serves as a buffer which is able to decrease water activity by reducing free water in contact with the superoxide dismutase and the catalase in the granule. If the moisture absorbing compound is added to the core, it is important that there is excessive buffer capacity to remove the water present after application of the inner salt layer. In one embodiment, the moisture absorbing compound has a water uptake of more than 3%, such as more than 5%, such as more than 10% water uptake. The water uptake is found as the equilibrium water uptake at 25° C. and 70% relative humidity after one week. The amount of moisture absorbing compound added to the granule is more than 1%, more than 2%, more than 5%, or more than 10% w/w of the granule.

The moisture absorbing compound may be either organic or inorganic compounds and may be selected from, but is not limited to, the group consisting of flour, starch, corn cob products, cellulose and silica gel.

The granule may comprise additional materials such as process aids, fillers, fibre materials, stabilizing agents, solubilising agents, suspension agents, viscosity regulating agents, light spheres, plasticizers, salts, lubricants and fragrances. Process aids may e.g. be provided as powdering and may e.g. be CaCO₃, talcum and/or kaolin. Suitable fillers are water soluble and/or insoluble inorganic salts such as finely ground alkali sulfate, alkali carbonate and/or alkali chloride, clays such as kaolin (e.g. SPESWHITE™, English China Clay), bentonites, talcs, zeolites, chalk, calcium carbonate and/or silicates. Typical fillers are di-sodium sulfate and calcium-lignosulphonate. Stabilising or protective agents are such as conventionally used in the field of granulation. Stabilising or protective agents may fall into several categories: alkaline or neutral materials, reducing agents, antioxidants and/or salts of first transition series metal ions. Each of these may be used in conjunction with other protective agents of the same or different categories. Examples of alkaline protective agents are alkali metal silicates, carbonates or bicarbonates. Examples of reducing protective agents are salts of sulfite, thiosulfite, thiosulfate or MnSO₄ while examples of antioxidants are methionine, butylated hydroxytoluene (BHT) or butylated hydroxyanisol (BHA). In particular stabilising agents may be salts of thiosulfates, e.g. sodium thiosulfate or methionine. Still other examples of useful stabilizers are gelatine, urea, sorbitol, glycerol, casein, Poly vinyl pyrrolidone (PVP), hydroxypropylmethylcellulose (HPMC), carboxymethyl cellulose (CMC), hydroxyethylcellulose (HEC), powder of skimmed milk and/or edible oils, such as soy oil or canola oil. Particular stabilizing agents in feed granules are a lactic acid source or starch. A preferred lactic acid source is corn steep liquor. It is also well known in the art that enzyme substrates such as starch, lipids, proteins etc can act as stabilizers for enzymes.

Superoxide Dismutase

The polypeptide of the invention having superoxide dismutase activity may be of fungal origin. In one aspect, the superoxide dismutase is of fungal origin. In a further aspect, the superoxide dismutase of the invention is a superoxide dismutase obtainable from a fungus selected from the group consisting of Trichoderma reesei, Aspergillus versicolor, Aspergillus deflectus, Aspergillus egyptiacus, Westerdykella sp. AS85-2, Aspergillus sp. XZ2669, Preussia terricola, Kionochaeta sp., Metapochonia bulbillosa, Xylomelasma sp. XZ0718, Preussia flanaganii, Cladobottyum sp., Westerdykella sp-46156, Trichoderma hamatum, Mycothermus thermophilus, Cephalotrichiella penicillate, Chaetomium megalocarpum, Chaetomium thermophilum var. thermophilum, Humicola hyalothermophila, Subramaniula anamorphosa, Sphingobacterium sp. T2, Trichoderma rossicum, Trichoderma lixii, Trichoderma sp-54723, Aspergillus niveus, Aspergillus templicola, Pochonia chlamydosporia var. spinulospora, Trichoderma sp-44174, Trichoderma rossicum, Trichoderma sp-54723, Trichoderma sp-44174, Metapochonia suchlasporia, Metarhizium marquandii, Diaporthe nobilis, Tolypocladium sp. XZ2627, Aspergillus japonicus, Metarhizium sp. XZ2431, Armillaria ostoyae, Trichoderma spirale, Aspergillus elegans, Trichoderma sinuosum, Trichoderma virens, Trichoderma harzianum, Fusicolla acetilerea, Plectosphaerella sp. 1-29, Mariannaea punicea, Penicillium oxalicum, Colletotrichum sp-71086, Aspergillus sp. nov. XZ3202, Trichoderma parapiluliferum, Aspergillus sp. nov. XZ3202, Mucor sp. XZ2651, Rhizomucor miehei, Mucor sp. XZ2651, Amphisphaeriaceae-sp 43674, Humicola fuscoatra and Valsaria rubricosa.

In one aspect, the isolated polypeptide having superoxide dismutase activity is selected from the group consisting of:
a. a polypeptide from Trichoderma reesei from having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 1;
b. a polypeptide from Aspergillus japonicus having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 2;
c. a polypeptide from Aspergillus templicola having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 3;
d. a polypeptide from Diaporthe nobilis having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 4; and
e. a polypeptide from Armillaria ostoyae having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 5.

In one aspect, the isolated polypeptide having superoxide dismutase activity is a polypeptide from Trichoderma reesei from having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 1.

In one aspect, the isolated polypeptide having superoxide dismutase activity is a polypeptide from Aspergillus japonicus having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 2.

In one aspect, the isolated polypeptide having superoxide dismutase activity is a polypeptide from Aspergillus templicola having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 3.

In one aspect, the isolated polypeptide having superoxide dismutase activity is a polypeptide from Diaporthe nobilis having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 4.

In one aspect, the isolated polypeptide having superoxide dismutase activity is a polypeptide from Armillaria ostoyae having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 5

Catalase

The polypeptide of the invention having catalase activity may be of fungal origin. In one aspect, the catalase is of fungal origin. In a further aspect, the catalase of the invention is a polypeptide having catalase activity which is obtainable from a source selected from the group consisting of Aspergillus niger, Thermoascus aurantiacus, Aspergillus lentulus, Scytalidium thermophilum, Talaromyces stipitatus, Malbranchea cinnamomea, Crassicarpon thermophilum, Penicillium emersonii, Aspergillus versicolor, Thermomucor indicae-seudaticae, Aspergillus fumigatus, Thermothelomyces thermophilus, Curvularia verruculosa, Mycothermus thermophilus, Penicillium oxalicum, Humicola hyalothermophila, Thermoascus crustaceus, Thielavia australiensis, Thielavia hyrcaniae and Neurospora crassa.

In a preferred embodiment, the catalase is a polypeptide having catalase activity which is obtainable from a source selected from the group consisting of Aspergillus niger, Scytalidium thermophilum and Thermoascus aurantiacus, more preferably from Aspergillus niger and Thermoascus aurantiacus.

In one aspect, the isolated polypeptide having catalase activity is selected from the group consisting of:
a. a polypeptide from Thermoascus aurantiacus having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 6;
b. a polypeptide from Thermoascus aurantiacus having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 7;
c. a polypeptide from Thermoascus aurantiacus having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 8;
d. a polypeptide from Thermoascus aurantiacus having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 9;
e. a polypeptide from Thermoascus aurantiacus having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 10; and
f. a polypeptide from Thermoascus aurantiacus having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 11.

In one aspect, the isolated polypeptide having catalase activity is a polypeptide from Thermoascus aurantiacus having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 6.

In one aspect, the isolated polypeptide having catalase activity is a polypeptide from Thermoascus aurantiacus having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 7.

In one aspect, the isolated polypeptide having catalase activity is a polypeptide from Thermoascus aurantiacus having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 8.

In one aspect, the isolated polypeptide having catalase activity is a polypeptide from Thermoascus aurantiacus having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 9 In one aspect, the isolated polypeptide having catalase activity is a polypeptide from Thermoascus aurantiacus having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 10.

In one aspect, the isolated polypeptide having catalase activity is a polypeptide from Thermoascus aurantiacus having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 11.

The Layer Surrounding the Core

The granule composition of the invention optionally has a layer surrounding the core. In one aspect of the invention the layer surrounding the core is a hydrophobic coating material, in a further aspect the layer is a wax coating. In a yet further aspect the wax coating comprises a wax which is selected from the group consisting of: castor oil, hydrogenated castor oil, palm kernel oil, hydrogenated palm kernel oil, palm oil, hydrogenated palm oil, hydrogenated cotton seeds, soy bean oil, hydrogenated soy bean oil, rapeseed oil, hydrogenated rapeseed oil, a blend of hydrogenated and unhydrogenated vegetable oil, 12-hyroxystearic acid, microcrystalline wax, high-melting paraffin waxes and mixtures thereof.

The layer surrounding the core, may in a particular embodiment of the present invention contribute between 10-30% w/w of the granule, such as between 15-30% w/w, or between 20-30% w/w of the granule. In one embodiment, the layer surrounding the core contributes about 24% w/w of the granule.

In a particular embodiment of the present invention the amount of hydrophobic coating material in the layer surrounding the core of the granule constitutes at least 60% w/w of the layer surrounding the core.

In a particular embodiment of the present invention the amount of hydrophobic coating material in the layer surrounding the core of the granules in the feed composition, such as e.g. steam treated feed compositions, constitutes at least 60% w/w of the layer surrounding the core.

In a particular embodiment of the present invention the amount of hydrophobic coating material in the layer surrounding the core of the granules to be used for feed compositions, such as e.g. steam treated feed compositions, constitutes at least 60% w/w of the layer surrounding the core.

To be able to provide acceptable protection, the layer surrounding the core preferably has a certain thickness. In a particular embodiment of the present invention the layer surrounding the core is at least 7 μm thick. In a more particular embodiment the thickness of the layer surrounding the core is at least 10 μm. In an even more particular embodiment the thickness of the layer surrounding the core is between 7-20 μm. In a most particular embodiment the thickness of the layer surrounding the core is between 10-20 μm. In a most particular embodiment the thickness of the layer surrounding the core is between 12-18 μm. In a particular embodiment of the present invention the thickness of the layer surrounding the core is below 21 μm. In a more particular embodiment the thickness of the layer surrounding the core is below 20 μm. In an even more particular embodiment the total thickness of the layer surrounding the core is below 18 μm.

In a particular embodiment of the present invention the thickness of the layer surrounding the core of the granules to be used for feed compositions, such as e.g. the steam treated pelletized feed composition, is at least 7 μm. In another particular embodiment of the present invention the thickness of the layer surrounding the core of the granules to be used for feed compositions, such as e.g. the steam treated pelletized feed composition, is at least 10 μm.

The layer surrounding the core should encapsulate the core by forming a substantially continuous layer, i.e. a layer surrounding the core having few or no holes, so that the core it is encapsulating has few or no uncoated areas. The layer surrounding the core should in a preferred embodiment be homogenous in thickness.

Referring to the hydrophobic coating material in the layer surrounding the core it can either be one particular hydrophobic coating material or a mixture of hydrophobic coating materials.

The hydrophobic coating material may include oils and/or waxes, including, without limitations, hydrogenated vegetable oils such as hydrogenated castor oil, hydrogenated palm kernel oil, hydrogenated palm oil, hydrogenated cotton seeds, hydrogenated soy bean oil and/or hydrogenated rapeseed oil, a blend of hydrogenated and unhydrogenated vegetable oil, 12-hyroxystearic acid, microcrystalline wax such as Cerit HOT, and high-melting paraffin waxes such as Mekon White.

Further hydrophobic coating materials included in the invention are combinations with water immiscible liquids or low melting point hydrophobic solids that produce a mixture with a reduced melting point. These include waxes, C26 and higher, paraffin waxes, cholesterol, fatty alcohols, such as cetyl alcohol, mono-, di- and/or triglycerides of animal and vegetable origin such as hydrogenated ox tallow, hydrogenated fat, hydrogenated castor oil, fat derivatives such as fatty acids, soaps, esters, hydrophobic starches such as ethyl cellulose, lecithin. The waxes may be of natural origin, meaning they may be animal, vegetable or mineral. Animal waxes include, without limitation, beeswax, lanolin, shellac wax and Chinese insect wax. Vegetable wax includes, without limitation, carnauba, candelilla, bayberry and sugar cane waxes. Mineral waxes include, without limitation, fossil or earth waxes including ozokerite, ceresin and montan or petroleum waxes, including paraffin and microcrystalline waxes. Alternatively the waxes may be synthetic or mixtures of natural and synthetic waxes. For example, synthetic or mixtures of natural and synthetic waxes may include low molecular weight partially oxidized polyethylene, which may be preferentially co-melted with paraffin. The fatty derivatives may be either fatty acids, fatty acid amides, fatty alcohols, fatty esters or mixtures of these. The acid amide may be stearamide. Sterols or long chain sterol esters may also be such as cholesterol or ergosterol.

A preferred hydrophobic coating material is palm oil or hydrogenated palm oil.

The Feed Composition

The granule of the present invention is suitable for use in animal feed compositions. The granule is mixed with feed substances. The characteristics of the granule allows its use as a component of a composition which is well suited as an animal feed, which is steam treated and subsequently pelletized.

The term feed or feed composition means any compound, preparation, mixture, or composition

The feed of the present invention may comprise vegetable proteins. The term vegetable proteins as used herein refers to any compound, composition, preparation or mixture that includes at least one protein derived from or originating from a vegetable, including modified proteins and protein-derivatives. In particular embodiments, the protein content of the vegetable proteins is at least 10, 20, 30, 40, 50, or 60% (w/w).

Vegetable proteins may be derived from vegetable protein sources, such as legumes and cereals, for example materials from plants of the families Fabaceae (Leguminosae), Cruciferaceae, Chenopodiaceae, and Poaceae, such as soy bean meal, lupin meal and rapeseed meal.

In a particular embodiment, the vegetable protein source is material from one or more plants of the family Fabaceae, e.g. soybean, lupine, pea, or bean.

In another particular embodiment, the vegetable protein source is material from one or more plants of the family Chenopodiaceae, e.g. beet, sugar beet, spinach or quinoa.

Other examples of vegetable protein sources are rapeseed, and cabbage.

Soybean is a preferred vegetable protein source.

Other examples of vegetable protein sources are cereals such as barley, wheat, rye, oat, maize (corn), rice, and sorghum.

Suitable animal feed additives are enzyme inhibitors, fat-soluble vitamins, water soluble vitamins, trace minerals and macro minerals.

Further, optional, feed-additive ingredients are colouring agents, aroma compounds, stabilisers, antimicrobial peptides, and/or at least one other enzyme selected from amongst phytases EC 3.1.3.8 or 3.1.3.26; xylanases EC 3.2.1.8; galactanases EC 3.2.1.89; and/or beta-glucanases EC 3.2.1.4.

Examples of anti microbial peptides (AMP's) are CAP18, Leucocin A, Tritrpticin, Protegrin-1, Thanatin, Defensin, Ovispirin such as Novispirin (Robert Lehrer, 2000), and variants, or fragments thereof which retain antimicrobial activity.

Examples of anti fungal polypeptides (AFP's) are the Aspergillus giganteus, and Aspergillus niger peptides, as well as variants and fragments thereof which retain antifungal activity, as disclosed in WO 94/01459 and PCT/DK02/00289.

Usually fat- and water-soluble vitamins, as well as trace minerals form part of a so-called premix intended for addition to the feed, whereas macro minerals are usually separately added to the feed.

The following are non-exclusive lists of examples of these components:

Examples of fat-soluble vitamins are vitamin A, vitamin D3, vitamin E, and vitamin K, e.g. vitamin K3.

Examples of water-soluble vitamins are vitamin B12, biotin and choline, vitamin B1, vitamin B2, vitamin B6, niacin, folic acid and panthothenate, e.g. Ca-D-panthothenate.

Examples of trace minerals are manganese, zinc, iron, copper, iodine, selenium, and cobalt.

Examples of macro minerals are calcium, phosphorus and sodium.

In still further particular embodiments, the animal feed composition of the invention contains 0-80% maize; and/or 0-80% sorghum; and/or 0-70% wheat; and/or 0-70% Barley; and/or 0-30% oats; and/or 0-40% soybean meal; and/or 0-10% fish meal; and/or 0-20% whey.

Preparation

Preparation of the Granule Core

The core of the granule of the invention may comprise a superoxide dismutase and a catalase in the form of concentrated dry matter. In one embodiment, the concentrated dry matter is prepared by spray drying.

Methods for preparing the core can be found in Handbook of Powder Technology; Particle size enlargement by C. E. Capes; Volume 1; 1980; Elsevier. Preparation methods include known feed and granule formulation technologies, i.e.:
a) Spray dried products, wherein a liquid superoxide dismutase and catalase-containing solution is atomized in a spray drying tower to form small droplets which during their way down the drying tower dry to form a superoxide dismutase and catalase-containing particulate material. Very small particles can be produced this way (Michael S. Showell (editor); Powdered detergents; Surfactant Science Series; 1998; vol. 71; page 140-142; Marcel Dekker).
b) Layered products, wherein the superoxide dismutase and catalase are coated as a layer around a pre-formed inert core particle, wherein an superoxide dismutase and catalase-containing solution is atomized, typically in a fluid bed apparatus wherein the pre-formed core particles are fluidized, and the superoxide dismutase and catalase-containing solution adheres to the core particles and dries up to leave a layer of dry superoxide dismutase and catalase on the surface of the core particle. Particles of a desired size can be obtained this way if a useful core particle of the desired size can be found. This type of product is described in e.g. WO 97/23606
c) Absorbed core particles, wherein rather than coating the superoxide dismutase and catalase as a layer around the core, the superoxide dismutase and catalase are absorbed onto and/or into the surface of the core. Such a process is described in WO 97/39116.
d) Extrusion or pelletized products, wherein a superoxide dismutase and catalase-containing paste is pressed to pellets or under pressure is extruded through a small opening and cut into particles which are subsequently dried. Such particles usually have a considerable size because of the material in which the extrusion opening is made (usually a plate with bore holes) sets a limit on the allowable pressure drop over the extrusion opening. Also, very high extrusion pressures when using a small opening increase heat generation in the superoxide dismutase and catalase paste, which is harmful to the superoxide dismutase and catalase. (Michael S. Showell (editor); Powdered detergents; Surfactant Science Series; 1998; vol. 71; page 140-142; Marcel Dekker)
e) Prilled products, wherein an active powder is suspended in molten wax and the suspension is sprayed, e.g. through a rotating disk atomiser, into a cooling chamber where the droplets quickly solidify (Michael S. Showell (editor); Powdered detergents; Surfactant Science Series; 1998; vol. 71; page 140-142; Marcel Dekker). The product obtained is one wherein the superoxide dismutase and catalase are uniformly distributed throughout an inert material instead of being concentrated on its surface. Also U.S. Pat. Nos. 4,016,040 and 4,713,245 are documents relating to this technique
f) Mixer granulation products, wherein an active-containing liquid is added to a dry powder composition of conventional granulating components. The liquid and the powder in a suitable proportion are mixed and as the moisture of the liquid is absorbed in the dry powder, the components of the dry powder will start to adhere and agglomerate and particles will build up, forming granulates comprising the superoxide dismutase and catalase. Such a process is described in U.S. Pat. No. 4,106,991 (NOVO NORDISK) and related documents EP 170360 B1 (NOVO NORDISK), EP 304332 B1 (NOVO NORDISK), EP 304331 (NOVO NORDISK), WO 90/09440 (NOVO NORDISK) and WO 90/09428 (NOVO NORDISK). In a particular product of this process wherein various high-shear mixers can be used as granulators, granulates consisting of enzyme as superoxide dismutase and catalase, fillers and binders etc. are mixed with cellulose fibres to reinforce the particles to give the so-called T-granulate. Reinforced particles, being more robust, release less enzymatic dust.
g) Size reduction, wherein the cores are produced by milling or crushing of larger particles, pellets, tablets, briquettes etc. containing the active material. The wanted core particle fraction is obtained by sieving the milled or crushed product. Over and undersized particles can be recycled. Size reduction is described in (Martin Rhodes (editor); Principles of Powder Technology; 1990; Chapter 10; John Wiley & Sons).
h) Fluid bed granulation. Fluid bed granulation involves suspending particulates in an air stream and spraying a liquid onto the fluidized particles via nozzles. Particles hit by spray droplets get wetted and become tacky. The tacky particles collide with other particles and adhere to them and form a granule.
i) The cores may be subjected to drying, such as in a fluid bed drier. Other known methods for drying granules in the feed or enzyme industry can be used by the skilled person. The drying preferably takes place at a product temperature of from 25 to 90° C. For some superoxide dismutase and/or catalases it is important the cores comprising the superoxide dismutase and catalase contain a low amount of water before coating with the hydrophobic layer surrounding the core. If water sensitive superoxide dismutase and catalases are coated before excessive water is removed, it will be trapped within the core and it may affect the activity of the superoxide dismutase and catalase negatively. After drying, the cores preferably contain 0.1-10 w/w water.

Preparation of the Layer Surrounding the Core

Conventional coatings and methods as known to the art may suitably be used as the layer surrounding the core, such as the coatings described in Danish PA 2002 00473, WO 89/08694, WO 89/08695, 270 608 B1 and/or WO 00/01793. Other examples of conventional coating materials may be found in U.S. Pat. No. 4,106,991, EP 170360, EP 304332, EP 304331, EP 458849, EP 458845, WO 97/39116, WO 92/12645A, WO 89/08695, WO 89/08694, WO 87/07292, WO 91/06638, WO 92/13030, WO 93/07260, WO 93/07263, WO 96/38527, WO 96/16151, WO 97/23606, WO 01/25412, WO 02/20746, WO 02/28369, U.S. Pat. Nos. 5,879,920, 5,324,649, 4,689,297, 6,348,442, EP 206417, EP 193829, DE 4344215, DE 4322229 A, DE 263790, JP 61162185 A and/or JP 58179492.

The coating may be prepared by the same method as mentioned above in the section “Preparation of the core”.

The granules obtained can be subjected to rounding off (e.g. spheronisation), such as in a Marumeriser™, or compaction.

The granules can be dried, such as in a fluid bed drier. Other known methods for drying granules in the feed or enzyme industry can be used by the skilled person. The drying preferably takes place at a product temperature of from 25 to 90° C.

Manufacturing of Feed Pellets

In the manufacturing of feed pellets it is preferred to involve steam treatment prior to pelleting, a process called conditioning. In the subsequent pelleting step the feed is forced through a die and the resulting strands are cut into suitable pellets of variable length. During this conditioning step the process temperature may rise to 60-100° C.

The feed mixture is prepared by mixing the granules comprising the superoxide dismutase and catalase with desired feed components. The mixture is led to a conditioner e.g. a cascade mixer with steam injection. The feed is in the conditioner heated up to a specified temperature, 60-100° C., e.g. 60° C., 70° C., 80° C., 90° C. or 100° C. by injecting steam, measured at the outlet of the conditioner. The residence time can be variable from seconds to minutes and even hours. Such as 5 seconds, 10 seconds, 15 seconds, 30 seconds, 1 minute, 2 minutes, 5 minutes, 10 minutes, 15 minutes, 30 minutes and 1 hour. In a particular embodiment of the present invention the temperature is 100° C. and the residence time is 60 seconds.

In a particular embodiment of the present invention the process temperature during steam treatment is at least 60° C. In a more particular embodiment of the present invention the process temperature during steam treatment is at least 70° C. In an even more particular embodiment of the present invention the process temperature during steam treatment is at least 80° C. In a most particular embodiment of the present invention the process temperature during steam treatment is at least 90° C.

From the conditioner the feed is led to a press e.g. a Simon Heesen press, and pressed to pellets with variable length e.g. 15 mm. After the press the pellets are placed in an air cooler and cooled for a specified time e.g. 15 minutes.

A particular embodiment of the present invention is a method for manufacturing a feed composition comprising the steps of:
i. mixing feed components with granules comprising a core, an inner salt layer and a layer surrounding the core wherein the core comprises an superoxide dismutase and catalase,
ii. steam treating said composition (i), and
iii. pelleting said composition (ii).

In an embodiment, the superoxide dismutase present in the core of the granules has retained at least 75% of the activity of the superoxide dismutase in the core of the granules after steam pelleting the feed at 85 degrees Celsius compared to the activity before steam pelleting. In a further embodiment, the superoxide dismutase present in the core of the granules has retained at least 75% of the activity of the superoxide dismutase in the core of the granules after steam pelleting at 90 degrees Celsius compared to the activity before steam pelleting. In a further embodiment, the superoxide dismutase present in the core of the granules has retained at least 75% of the activity of the superoxide dismutase in the core of the granules after steam pelleting at 95 degrees Celsius compared to the activity before steam pelleting. In a yet further embodiment, the superoxide dismutase present in the core of the granules has retained at least 80% of the activity of the superoxide dismutase in the core of the granules after steam pelleting at 85 degrees Celsius compared to the activity before steam pelleting. In a yet further embodiment, the superoxide dismutase present in the core of the granules has retained at least 80% of the activity of the superoxide dismutase in the core of the granules after steam pelleting at 90 degrees Celsius compared to the activity before steam pelleting. In a yet further embodiment, the superoxide dismutase present in the core of the granules has retained at least 80% of the activity of the superoxide dismutase in the core of the granules after steam pelleting at 95 degrees Celsius compared to the activity before steam pelleting. In a still further embodiment, the superoxide dismutase present in the core of the granules has retained at least 85% of the activity of the superoxide dismutase in the core of the granules after steam pelleting at 85 degrees Celsius compared to the activity before steam pelleting. In a still further embodiment, the superoxide dismutase present in the core of the granules has retained at least 85% of the activity of the superoxide dismutase in the core of the granules after steam pelleting at 90 degrees Celsius compared to the activity before steam pelleting. In a still further embodiment, the superoxide dismutase present in the core of the granules has retained at least 85% of the activity of the superoxide dismutase in the core of the granules after steam pelleting at 95 degrees Celsius compared to the activity before steam pelleting.

In an embodiment, the catalase present in the core of the granules has retained at least 75% of the activity of the catalase in the core of the granules after steam pelleting the feed at 85 degrees Celsius compared to the activity before steam pelleting. In a further embodiment, the catalase present in the core of the granules has retained at least 75% of the activity of the catalase in the core of the granules after steam pelleting at 90 degrees Celsius compared to the activity before steam pelleting. In a further embodiment, the catalase present in the core of the granules has retained at least 75% of the activity of the catalase in the core of the granules after steam pelleting at 95 degrees Celsius compared to the activity before steam pelleting. In a yet further embodiment, the catalase present in the core of the granules has retained at least 80% of the activity of the catalase in the core of the granules after steam pelleting at 85 degrees Celsius compared to the activity before steam pelleting. In a yet further embodiment, the catalase present in the core of the granules has retained at least 80% of the activity of the catalase in the core of the granules after steam pelleting at 90 degrees Celsius compared to the activity before steam pelleting. In a yet further embodiment, the catalase present in the core of the granules has retained at least 80% of the activity of the catalase in the core of the granules after steam pelleting at 95 degrees Celsius compared to the activity before steam pelleting. In a still further embodiment, the catalase present in the core of the granules has retained at least 85% of the activity of the catalase in the core of the granules after steam pelleting at 85 degrees Celsius compared to the activity before steam pelleting. In a still further embodiment, the catalase present in the core of the granules has retained at least 85% of the activity of the catalase in the core of the granules after steam pelleting at 90 degrees Celsius compared to the activity before steam pelleting. In a still further embodiment, the catalase present in the core of the granules has retained at least 85% of the activity of the catalase in the core of the granules after steam pelleting at 95 degrees Celsius compared to the activity before steam pelleting.

In an embodiment, the superoxide dismutase and the catalase present in the core of the granules has retained at least 75% of the activity of the superoxide dismutase and the catalase in the core of the granules after steam pelleting the feed at 85 degrees Celsius compared to the activity before steam pelleting. In a further embodiment, the superoxide dismutase and the catalase present in the core of the granules has retained at least 75% of the activity of the superoxide dismutase and the catalase in the core of the granules after steam pelleting at 90 degrees Celsius compared to the activity before steam pelleting. In a further embodiment, the superoxide dismutase and the catalase present in the core of the granules has retained at least 75% of the activity of the superoxide dismutase and the catalase in the core of the granules after steam pelleting at 95 degrees Celsius compared to the activity before steam pelleting. In a yet further embodiment, the superoxide dismutase and the catalase present in the core of the granules has retained at least 80% of the activity of the superoxide dismutase and the catalase in the core of the granules after steam pelleting at 85 degrees Celsius compared to the activity before steam pelleting. In a yet further embodiment, the superoxide dismutase and the catalase present in the core of the granules has retained at least 80% of the activity of the superoxide dismutase and the catalase in the core of the granules after steam pelleting at 90 degrees Celsius compared to the activity before steam pelleting. In a yet further embodiment, the superoxide dismutase and the catalase present in the core of the granules has retained at least 80% of the activity of the superoxide dismutase and the catalase in the core of the granules after steam pelleting at 95 degrees Celsius compared to the activity before steam pelleting. In a still further embodiment, the superoxide dismutase and the catalase present in the core of the granules has retained at least 85% of the activity of the superoxide dismutase and the catalase in the core of the granules after steam pelleting at 85 degrees Celsius compared to the activity before steam pelleting. In a still further embodiment, the superoxide dismutase and the catalase present in the core of the granules has retained at least 85% of the activity of the superoxide dismutase and the catalase in the core of the granules after steam pelleting at 90 degrees Celsius compared to the activity before steam pelleting. In a still further embodiment, the superoxide dismutase and the catalase present in the core of the granules has retained at least 85% of the activity of the superoxide dismutase and the catalase in the core of the granules after steam pelleting at 95 degrees Celsius compared to the activity before steam pelleting.

Methods of Improving Animal Health or Growth

The granules of the invention may be used for improving animal health and/or growth.

In one aspect, the invention relates to a method of improving one or more performance parameters in an animal comprising administering to the animal an animal feed or animal feed additive comprising the granule of the invention, wherein the one or more performance parameters is selected from the group consisting of the European Production Efficiency Factor (EPEF), Feed Conversion Ratio (FCR), Growth Rate (GR), Body Weight Gain (WG), Mortality Rate (MR) and Flock Uniformity (FU).

In a further aspect, the invention relates to a method of improving or enhancing immune response and/or reducing inflammation and/or for the modulation of the gut flora in an animal comprising administering to the animal an animal feed or animal feed additive comprising the granule of the invention. A related aspect of the invention is directed to the prophylactic care or management, reduction or prevention of inflammation in the intestinal tract of a monogastric animal.

In a further aspect, the invention relates to a method of reducing or eliminating the use of antibiotics administered to animal feed, comprising administering to the animal an animal feed or animal feed additive comprising the granule of the invention.

In a further aspect, the invention relates to a method of reducing cellular markers of reactive oxygen species or free radicals in animal body comprising administering to the animal an animal feed or animal feed additive comprising the granule of the invention.

A further aspect of the invention is directed to the prophylactic care or management, reduction or prevention of oxidative stress in a monogastric animal comprising administrating to said animal the granule of the invention. Oxidative stress is a disturbance between antioxidant/oxidant status in favor of excessive generation, or slower removal of free radicals, such as reactive oxygen species (ROS). Excessive ROS content leads to damage of proteins, lipids and nucleic acids, with consequent loss of their biological functions and subsequent tissue injury. Oxidative stress has been linked to initiation and progression of several infectious diseases. Accordingly, a further aspect of the invention is the prophylactic care or management of infectious diseases in monogastric animal comprising administrating to said animal the granule of the invention. The administration is typically by means of feeding said animal a feed additive comprising the granule.

In respect to the improvement of one or more performance parameters, the inventions is particularly characterized in that the EPEF and/or FCR and/or GR and/or WG is improved by at least 1% and that the MR is reduced by at least 1%.

The term animal includes all animals. Examples of animals are non-ruminants, and ruminants, such as cows, sheep and horses. The animal is in one aspect a mono-gastric animal, e.g. pigs or swine (including, but not limited to, piglets, growing pigs, and sows); poultry (including but not limited to poultry, turkey, duck, quail, guinea fowl, goose, pigeon, squab, chicken, broiler, layer, pullet and chick); pets (including but not limited to cats and dogs); fish (including but not limited to amberjack, arapaima, barb, bass, bluefish, bocachico, bream, bullhead, cachama, carp, catfish, catla, chanos, char, cichlid, cobia, cod, crappie, dorada, drum, eel, goby, goldfish, gourami, grouper, guapote, halibut, java, labeo, lai, loach, mackerel, milkfish, mojarra, mudfish, mullet, paco, pearlspot, pejerrey, perch, pike, pompano, roach, salmon, sampa, sauger, sea bass, seabream, shiner, sleeper, snakehead, snapper, snook, sole, spinefoot, sturgeon, sunfish, sweetfish, tench, terror, tilapia, trout, tuna, turbot, vendace, walleye and whitefish); and crustaceans (including but not limited to shrimps and prawns). In a more preferred embodiment, the animal is selected from the group consisting of swine, poultry, crustaceans and fish. In an even more preferred embodiment, the animal is selected from the group consisting of swine, piglet, growing pig, sow, chicken, broiler, layer, pullet and chick, typically wherein the animal has experienced heat stress, cold stress, nutritional stress and/or oxidative stress.

A further aspect of the invention is directed to a method of feeding poultry or pigs comprising adding the animal feed additive of the invention to a raw feed material.

A further aspect of the invention is directed to a method of feeding an animal, wherein the animal feed or animal feed additive comprises the granule of the invention and further comprises one or more components selected from the list consisting of:
i. one or more carriers;
ii. one or more microbes;
iii. one or more vitamins;
iv. one or more minerals;
v. one or more amino acids;
vi. one of more organic acids;
vii. and one or more other feed ingredients.

### PREFERRED EMBODIMENTS

The following is a list of preferred embodiments further describing the invention:
1. A granule comprising a polypeptide having superoxide dismutase activity and a polypeptide having catalase activity, wherein the molar ratio between the polypeptide having superoxide dismutase activity and the polypeptide having catalase activity is greater than 5:1, such as at least 9:1, such as between 99:1 and 9:1.
2. The granule of embodiment 1, wherein the ratio between the polypeptide having superoxide dismutase activity and the polypeptide having catalase activity is between 74:1 and 9:1 such as between 49:1 and 9:1, 39:1 and 9:1, 39:1 and 19:1, 34:1 and 19:1 or 34:1 and 24:1.
3. The granule of embodiment 1, wherein the ratio between the polypeptide having superoxide dismutase activity and the polypeptide having catalase activity is about 29:1.
4. The granule of any one of the preceding embodiments, wherein the polypeptide having superoxide dismutase activity and the polypeptide having catalase activity are present as a mixture in the granule.
5. The granule of any of the preceding embodiments, wherein the granule comprises a core and a layer surrounding the core.
6. The granule of embodiment 5, wherein the polypeptide having superoxide dismutase activity and the polypeptide having catalase activity are present in the core of the granule.
7. The granule of any one of the preceding embodiments, wherein the amount of polypeptide having superoxide dismutase activity is between 1-10% w/w of the granule.
8. The granule of any one of the preceding embodiments, wherein the amount of polypeptide having superoxide dismutase activity is between 1-8% w/w of the granule, such as between 1-6% w/w, 1-5% w/w, 1-3,5% w/w, 1-3%, 1-2.5% or 1.5-2.5% w/w of the granule.
9. The granule of any one of the preceding embodiments, wherein the amount of polypeptide having superoxide dismutase activity is about 2% w/w of the granule.
10. The granule of any one of the preceding embodiments, wherein the amount of polypeptide having catalase activity is between 0.02-1.5% w/w of the granule.
11. The granule of any one of the preceding embodiments, wherein the amount of polypeptide having catalase activity is between 0.03-1.2% w/w of the granule, such as between 0.03-1.0%, 0.03-0.8%, 0.03-0.5%, 0.03-0.1%, 0.04-0.1%, 0.05-0.1%, 0.05-0.09% or 0.06%-0.08 w/w of the granule.
12. The granule of any one of the preceding embodiments, wherein the amount of polypeptide having catalase activity is about 0.1% w/w of the granule.
13. The granule of any one of the preceding embodiments, wherein the amount of polypeptide having catalase activity is about 0.07% w/w of the granule.
14. The granule of any one of the preceding embodiments, wherein the polypeptide having superoxide dismutase activity is of fungal origin.
15. The granule of any one of the preceding embodiments, wherein the polypeptide having superoxide dismutase (SOD) activity is selected from the group consisting of:
   a. a polypeptide from Trichoderma reesei from having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 1;
   b. a polypeptide from Aspergillus japonicus having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 2;
   c. a polypeptide from Aspergillus templicola having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 3;
   d. a polypeptide from Diaporthe nobilis having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 4; and
   e. a polypeptide from Armillaria ostoyae having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 5.
16. The granule of any one of the preceding embodiments, wherein the polypeptide having catalase activity is of fungal origin.
17. The granule of any one of the preceding embodiments, wherein the polypeptide having catalase is selected from the group consisting of:
   a. a polypeptide from Thermoascus aurantiacus having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 6;
   b. a polypeptide from Thermoascus aurantiacus having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 7;
   c. a polypeptide from Thermoascus aurantiacus having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 8;
   d. a polypeptide from Thermoascus aurantiacus having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 9;
   e. a polypeptide from Thermoascus aurantiacus having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 10; and
   f. a polypeptide from Thermoascus aurantiacus having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 11.
18. The granule of any one of the preceding embodiments, wherein the granules have a particle size which is between 50 μm-2000 μm.
19. The granule of any one of the preceding embodiments, wherein the granules have a particle size which is between 100 μm-1500 μm.
20. The granule of any one of the preceding embodiments, wherein the granules have a particle size which is between 100 μm-700 μm.
21. The granule of any one of embodiments 1 to 19, wherein the mean particle size of the granule is between 200 and 800 μm.
22. The granule of any one of the preceding embodiments, wherein the mean particle size of the granule is between 300 and 650 μm.
23. The granule of any one of the preceding embodiments, wherein the mean particle size of the granule is about 300 μm.
24. The granule of any one of embodiments 1 to 22, wherein the mean particle size of the granule is about 650 μm.
25. The granule of any one of embodiments 5 to 24, wherein the core comprises an inert particle.
26. The granule of any one of embodiments 5 to 25, wherein the core further comprises one or more ingredients selected from the group consisting of: one or more salts, one or more carriers, one or more particulate fillers and one or more granulation binders.
27. The granule of embodiment 26, wherein the salt is a stabilisator.
28. The granule of embodiment 26 or 27, wherein the salt is selected from the group consisting of: Na₂SO₄, K₂SO₄, KHSO₄, ZnSO₄, MgSO₄, CuSO₄, (NH₄)₂SO₄, Mg(CH₃COO)₂, Zn(CH₃COO)₂, magnesium acetate, and sodium citrate.
29. The granule of embodiment 28, wherein the salt is a hydrated salt such as magnesium sulfate heptahydrate (MgSO₄(7H₂O)), zinc sulfate heptahydrate (ZnSO₄(7H₂O)), sodium citrate dihydrate and/or magnesium acetate tetrahydrate.
30. The granule of any one of embodiments 26 to 29, wherein the salt is MgSO₄ or MgSO₄(7H₂O).
31. The granule of any one of embodiments 5 to 25, wherein the one or more ingredients in the core is selected from the group consisting of: cellulose, calcium carbonate, sodium sulfate, magnesium sulfate, zinc sulfate, dextrin, sucrose, kaolin, and a mixture of any of these.
32. The granule of any one of embodiments 5 to 31, wherein the layer surrounding the core is a hydrophobic coating layer
33. The granule of embodiment 32, wherein the layer surrounding the core is a wax coating.
34. The granule of embodiment 33, wherein the wax coating comprises a wax which is selected from the group consisting of: castor oil, hydrogenated castor oil, palm kernel oil, hydrogenated palm kernel oil, palm oil, hydrogenated palm oil, hydrogenated cotton seeds, soy bean oil, hydrogenated soy bean oil, rapeseed oil, hydrogenated rapeseed oil, a blend of hydrogenated and unhydrogenated vegetable oil, 12-hyroxystearic acid, microcrystalline wax, high-melting paraffin waxes and mixtures thereof.
35. The granule of any one of embodiments 5 to 34, wherein the layer surrounding the core comprises hydrogenated palm oil.
36. The granule of any one of embodiments 5 to 35, wherein the layer surrounding the core contributes between 10-30% w/w of the granule.
37. The granule of any one of embodiments 5 to 36, wherein the layer surrounding the core contributes between 15-30% w/w of the granule.
38. The granule of any one of embodiments 5 to 37, wherein the layer surrounding the core contributes between 20-30% w/w of the granule.
39. The granule of any one of embodiments 5 to 38, wherein the layer surrounding the core contributes about 24% w/w of the granule.
40. The granule of any one of embodiments 5 to 39, wherein the thickness of the layer surrounding the core is at least 7 μm.
41. The granule of any one of embodiments 5 to 40, wherein the thickness of the layer surrounding the core is between 7-20 μm.
42. The granule of any one of embodiments 5 to 41, wherein the thickness of the layer surrounding the core is between 10-20 μm.
43. The granule of any one of embodiments 5 to 42, wherein the thickness of the layer surrounding the core is between 12-18 μm.
44. The granule of any one of embodiments 5 to 43, wherein the thickness of the layer surrounding the core is about 15 μm.
45. The granule of any one of the preceding embodiments, wherein the granule further comprises a process aid.
46. The granule of embodiment 45, wherein the process aid is provided as powdering.
47. The granule of embodiment 46, wherein the process aid is CaCO₃, talcum and/or kaolin.
48. The granule of any one of the preceding embodiments, wherein the activity of the polypeptide having superoxide dismutase (SOD) activity present in the core of the granules after steam pelleting at 90 degrees Celsius is retained at least 65% compared to the activity of the polypeptide having superoxide dismutase (SOD) activity in the core of the granules before steam pelleting.
49. The granule of any one of the preceding embodiments, wherein the activity of the catalase present in the core of the granules after steam pelleting at 90 degrees Celsius is retained at least 65% compared to the activity of the catalase in the core of the granules before steam pelleting.
50. A process of preparing the granules of embodiments 1 to 49, comprising the steps of:
   a. preparing a core comprising a polypeptide having superoxide dismutase activity, a polypeptide having catalase activity and an inert material (e.g. salt, dextrin, cellulose)
   b. coating the core with a layer surrounding the core
51. The process of embodiment 50, wherein the granule is prepared in a mixer, a drum granulator, a fluid bed, a fluidized spray dryer, a spray fluidizer, a spray dryer or an extruder.
52. The process of any one of embodiments 50 to 51, wherein the core is prepared by drum granulating a mixture comprising the polypeptide having superoxide dismutase activity, the polypeptide having catalase activity and other ingredients of the core with a granulation fluid to form the granules.
53. The process of embodiment 52, wherein the other ingredients of the core are one or more ingredients selected from the group consisting of: one or more salts, one or more carriers, one or more particulate fillers and one or more granulation binders.
54. The process of embodiment 52, wherein the other ingredients of the core are one or more ingredients selected from the group consisting of: cellulose, calcium carbonate, sodium sulfate, magnesium sulfate, zinc sulfate, dextrin, sucrose, kaolin, and a mixture of any of these.
55. The process of any one of embodiments 52 to 54, wherein the granulation fluid is water
56. The process of any one of embodiments 50 to 55, wherein the layer surrounding the core is a hydrophobic coating comprising a mixture comprising a wax and an inorganic filler material such as kaolin or CaCO₃.
57. The process of any one of embodiments 50 to 56, wherein the process comprises the steps:
   a. loading one or more carriers into a fluid bed,
   b. spraying an aqueous solution comprising the polypeptide having superoxide dismutase activity, the polypeptide having catalase activity and optional other ingredients onto the carrier,
   c. drying the mixture in the fluid bed to obtain the core of the granule,
   d. spraying an aqueous solution of a coating material into the material in the fluid bed, and drying.
58. The process of any one of embodiments 50 to 56, wherein the process comprises the steps:
   a. contacting absorbent cores, capable of absorbing at least 5% w/w (based on the weight of the core) of water, with a liquid medium containing the polypeptide having superoxide dismutase activity and the polypeptide having catalase activity in dissolved and/or dispersed form, the amount of said liquid medium employed being such that substantially no attendant agglomeration of the resulting product occurs,
   b. at least partially removing volatile components of the liquid medium from said resulting product, and
   c. applying a coating to the granules.
59. An animal feed composition comprising feed components and the granule of any one of embodiments 1 to 49.
60. The animal feed composition of embodiment 59, wherein the feed components are selected from the group consisting of vegetable protein, fat-soluble vitamins, water soluble vitamins, trace minerals, macro minerals and combinations thereof.
61. A pelletized feed composition comprising any of the granules of any one of embodiments 1 to 49.
62. A steam treated feed composition comprising any of the granules of any one of embodiments 1 to 49.
63. A method for manufacturing an animal feed composition comprising the steps of:
   a. mixing feed components with the granules of any one of embodiments 1 to 49,
   b. steam treating the mixture of step (a), and
   c. optionally pelleting the steam treated mixture of step (b).
64. The use of any of the granules of embodiment 1 to 49 for steam treated pelletized feed compositions.
65. A composition comprising a polypeptide having superoxide dismutase activity and a polypeptide having catalase activity,
   wherein upon applying thermal stress to said composition, the activity of the polypeptide having catalase activity in the composition is higher than in a control composition having been applied said thermal stress, wherein said polypeptide having catalase activity is the only polypeptide in said control composition.
66. A co-granulate composition comprising a polypeptide having superoxide dismutase activity and a polypeptide having catalase activity wherein the activity of the polypeptide having catalase activity in the co-granulate composition is higher than in a granulate composition wherein said polypeptide having catalase activity is the only polypeptide, said catalase activity measured upon applying thermal stress to the said co-granulate composition and to said granulate composition.
67. A granule composition comprising a polypeptide having superoxide dismutase activity and a polypeptide having catalase activity, wherein the molar ratio between the polypeptide having superoxide dismutase activity and the polypeptide having catalase activity is greater than 5:1, such as at least 9:1, such as between 99:1 and 9:1.
68. A method of improving the stability of catalase comprising preparing a composition comprising said catalase and a polypeptide having superoxide dismutase activity.
69. A method of improving the stability of catalase comprising preparing a composition comprising said catalase and a polypeptide having superoxide dismutase activity, wherein upon applying thermal stress to said composition, the activity of the polypeptide having catalase activity in said composition is higher than in a control composition having been applied said thermal stress, wherein said polypeptide having catalase activity is the only polypeptide in said control composition.
70. The method according to embodiment 68 or 69 wherein the composition is prepared wherein the molar ratio between the polypeptide having superoxide dismutase activity and the catalase is greater than 5:1 such as at least 9:1.

### EXAMPLES

### Example 1 Granules Comprising Catalase

Granule 1 (Raw Granule)

A powder mixture with the following composition

| | | |
|---|---|---|
| 1200 | g | cellulose, Arbocel BC200 |
| 1950 | g | sucrose, CaCO3 |
| 10558 | g | ground Na₂SO₄ |

was granulated in a Lödige mixer FM 50
with a granulation fluid consisting of

| | | |
|---|---|---|
| 1200 | g | Sucrose |
| 196 | g | Catalase concentrate containing the polypeptide of |
| | | SEQ ID No: 6 |
| 1696 | g | water |

The granulation was carried out as described in U.S. Pat. No. 4,106,991, example 1. The granule was dried in a fluid bed dryer to a water content of less than 1% and sifted to obtain a product with the particle size between 250 and 1200 micrometers.

Granule 2 (Palm Oil Coating)

2.0 kg of above granule 1 was placed into a Lödige mixer L 5.

The following mixture was prepared for coating on the cores:

| | |
|---|---|
| 200 g | Melted hydrogenated palm oil |
| 460 g | Kaolin |

After coating, the final product was cooled in a MP 2 fluid bed.

Measurements of Stability

The stability of the granule was calculated on Granule 2.

Experimental Set-Up:

Duplicate samples of 15 g from batch at 40° C. & 50° C. temperature was stored in glass vials Ø35×75 mm with 28 mm collar. The vials were fitted with rubber sealed metal caps. The samples stored at 40° C./60% relative humidity were stored in similar vials containing 15 g granulate which were not sealed. After completion of the storage period all samples were stored refrigerated until analysis. Start samples were kept at −18° C. during the test period and were taken out and analyzed together with the test samples at different time points to minimize variation. The samples were analyzed for catalase enzyme activity in units per gram (U/g).

Measurements of Stability of the Granules

| | | % residual activity | | |
|---|---|---|---|---|
| | | 4 weeks/ | 4 weeks/ | 4 weeks/ |
| Formulation | Comprising | 40° C. | 50° C. | 40° C. & 60% RH |
| Product 1 | Granule 2 | 95 | 92 | 58 |

### Example 2 Granules Comprising Catalase

Granule 3 (Raw Granule)

A powder mixture with the following composition

| | | |
|---|---|---|
| 1200 | g | cellulose, Arbocel BC200 |
| 1950 | g | sucrose, CaCO3 |
| 10108 | g | ground Na₂SO₄ |

was granulated in a Lödige mixer FM 50
with a granulation fluid consisting of

| | | |
|---|---|---|
| 1200 | g | Sucrose |
| 450 | g | MgSO4•7H2O |
| 196 | g | Catalase concentrate containing the polypeptide of |
| | | SEQ ID No: 6 |
| 1696 | g | water |

The granulation was carried out as described in U.S. Pat. No. 4,106,991, example 1.

The granule was dried in a fluid bed dryer to a water content of less than 1% and sifted to obtain a product with the particle size between 250 and 1200 micrometers.

Granule 4 (Palm Oil Coating)

2.0 kg of above granule 3 was placed into a Lödige mixer L 5.

The following mixture was prepared for coating on the cores:

| | |
|---|---|
| 200 g | Melted hydrogenated palm oil |
| 460 g | Kaolin |

After coating the final product were cooled in a MP 2 fluid bed.

Measurements of Stability

The stability of the granules was calculated on Granule 4.

Experimental Set-Up:

Duplicate samples of 15 g from batch at 40° C. & 50° C. temperature was stored in glass vials Ø35×75 mm with 28 mm collar. The vials were fitted with rubber sealed metal caps. The samples stored at 40° C./60% relative humidity were stored in similar vials containing 15 g granulate which were not sealed. After completion of the storage period all samples were stored refrigerated until analysis. Start samples were kept at −18° C. during the test period and were taken out and analyzed together with the test samples at different time points to minimize variation.

Measurements of Stability of the Granules.

| | | % residual activity | | |
|---|---|---|---|---|
| | | 4 weeks/ | 4 weeks/ | 4 weeks/ |
| Formulation | Comprising | 40° C. | 50° C. | 40° C. & 60% RH |
| Product 2 | Granule 4 | 95 | 83 | 95 |

### Example 3 Granules Comprising Superoxide Dismutase

Granule 5 (Raw Granule)

A powder mixture with the following composition

| | | |
|---|---|---|
| 960 | g | cellulose, Arbocel BC200 |
| 1540 | g | dextrin, kaolin |
| 8867 | g | ground Na₂SO₄ |

was granulated in a Lödige mixer FM 50
with a granulation fluid consisting of

| | | |
|---|---|---|
| 480 | g | Dextrin |
| 1331 | g | Superoxide dismutase concentrate containing the polypeptide |
| | | of SEQ ID No: 1 |
| 242 | g | water |

The granulation was carried out as described in U.S. Pat. No. 4,106,991, example 1.

The granulated was dried in a fluid bed dryer to a water content of less than 1% and sifted to obtain a product with the particle size between 250 and 1200 micrometers.

Granule 6 (Palm Oil Coating)

2.0 kg of above granule 5 was placed into a Lödige mixer L 5.

The following mixture was prepared for coating on the cores:

| | |
|---|---|
| 200 g | Melted hydrogenated palm oil |
| 460 g | Kaolin |

After coating the final product were cooled in a MP 2 fluid bed.

Measurements of Stability

The stability of the granules was calculated on Granule 4.

Experimental Set-Up:

Duplicate samples of 15 g from batch at 40° C. & 50° C. temperature was stored in glass vials Ø35×75 mm with 28 mm collar. The vials were fitted with rubber sealed metal caps. The samples stored at 40° C./60% relative humidity were stored in similar vials containing 15 g granulate which were not sealed. After completion of the storage period all samples were stored refrigerated until analysis. Start samples were kept at −18° C. during the test period and were taken out and analyzed together with the test samples at different time points to minimize variation. The samples were analyzed for superoxide dismutase enzyme activity in units per gram (U/g).

Measurements of Stability of the Granules

| | | % residual activity |
|---|---|---|
| Formulation | Comprising | 4 weeks/50° C. |
| Product 3 | Granule 6 | 90 |

Measurements of Steam Stability

Granule 6 were steam treated at elevated temperature and at different retention times.

Experimental Set-Up:

Approximately 1 g enzyme granulate are heated up 95° C. in a furnace by injecting steam. The residence time in the conditioner was also varied from 90 seconds to 150 seconds. After the steam treatment the granules were placed in an air cooler and cooled for 15 minutes. The enzyme activity in the granules was measured before and after the steam treatment.

% residual activity

| Formulation | Comprising | 95° C./90sec | 95° C./150sec |
|---|---|---|---|
| Product 3 | Granule 6 | 95 | 60 |

### Example 4 Catalase and Superoxide Dismutase Co-Granule

Granule 7 (Raw Granule)

A powder mixture with the following composition

| | | |
|---|---|---|
| 960 | g | cellulose, Arbocel BC200 |
| 960 | g | kaolin |
| 957 | g | Spray dried superoxide dismutase |
| 8036 | g | ground Na₂SO₄ |

was granulated in a Lödige mixer FM 50
with a granulation fluid consisting of

| | | |
|---|---|---|
| 720 | g | Dextrin |
| 1926 | g | Superoxide dismutase concentrate containing the polypeptide |
| | | of SEQ ID No: 1 |
| 3 | g | Catalase concentrate containing the polypeptide of |
| | | SEQ ID No: 6 |

The granulation was carried out as described in U.S. Pat. No. 4,106,991, example 1.

The granulated was dried in a fluid bed dryer to a water content of less than 1% and sifted to obtain a product with the particle size between 250 and 1200 micrometers.

Granule 8 (Palm Oil Coating)

2.0 kg of above granule 7 was placed into a Lödige mixer L 5.

The following mixture was prepared for coating on the cores:

| | |
|---|---|
| 200 g | Melted hydrogenated palm oil |
| 460 g | Kaolin |

After coating the final product were cooled in a MP 2 fluid bed.

Measurements of Process Yield

The stability of the granules was calculated on granule 8.

Experimental Set-Up:

Duplicate samples of 15 g from batch at 40° C. & 50° C. temperature was stored in glass vials Ø35×75 mm with 28 mm collar. The vials were fitted with rubber sealed metal caps. The samples stored at 40° C./60% relative humidity were stored in similar vials containing 15 g granulate which were not sealed. After completion of the storage period all samples were stored refrigerated until analysis. Start samples were kept at −18° C. during the test period and were taken out and analyzed together with the test samples at different time points to minimize variation. The samples were analyzed for superoxide dismutase and catalase enzyme activity in units per gram (U/g).

Measurements of Stability of the Granules

| | | % residual activity | |
|---|---|---|---|
| Formulation | Comprising | 4 weeks/50° C. | |
| Product 4 | Granule 8 | 80 | SOD |

### Example 5 Catalase and Superoxide Dismutase Co-Granule

Granule 9 (Raw Granule)—Catalase Granulate

A powder mixture with the following composition

| | | |
|---|---|---|
| 1200 | g | Cellulose, Arbocel BC200 |
| 1200 | g | Kaolin |
| 11436 | g | ground Na₂SO₄ |

was granulated in a Lödige mixer FM 50
with a granulation fluid consisting of

| | | |
|---|---|---|
| 900 | g | Sucrose |
| 14 | g | Catalase concentrate |
| 1716 | g | Superoxide dismutase concentrate |
| 484 | g | water |

The granulation was carried out as described in U.S. Pat. No. 4,106,991, example 1.

The granulated was dried in a fluid bed dryer to a water content of less than 1% and sifted to obtain a product with the particle size between 250 and 850 micrometers.

Granule 10 (Salt Coating)

4.0 kg of above granule 9 was loaded into a Glatt Procell top-spray fluid bed.

The following mixture was coated onto the cores:

| | |
|---|---|
| 1400 g | Na2SO4 |
| 3430 g | Water |

After coating the granules were dried to a product temperature of 60° C.

### Example 6 Description of Steam Box

Granules 1 to 10 were subjected to a laboratory scale steaming box, where they were exposed to a temperature of 95° C. and 95% relative humidity, for a conditioning time of 90 sec

### Example 7 Steam Box Stability-Effect of Pelleting Conditions and Granulation Type on SOD Stability

Stability studies according to Example 6 were performed on SOD-containing granules prepared according to Example 4

TABLE 1a
SOD: SEQ ID NO 2

| | | SOD Activity | |
|---|---|---|---|
| | | (pre-thermal | Stability |
| Granule | Formulation | stress) | (activity %) |
| Raw granule | Cellulose/Dextrin/Kaolin/Na₂SO₄ | 3490 | 86% |
| Palm oil coating | Cellulose/Dextrin/Kaolin/Na₂SO₄ | 2470 | 86% |
| (30 wt %) | | | |
| Salt coating | Cellulose/Dextrin/Kaolin/Na₂SO₄ | 2900 | 75% |
| (33 wt %) | | | |
| Raw granule plus | Cellulose/Dextrin/Kaolin/Na₂SO₄/ | 7790 | 91% |
| MgSO₄•7H₂O | MgSO₄•7H₂O | | |
| Palm oil coating | Cellulose/Dextrin/Kaolin/Na₂SO₄/ | 5490 | 93% |
| plus MgSO₄•7H₂O | MgSO₄•7H₂O | | |
| Salt coating | Celllose/Dextrin/Kaolin/Na₂SO₄/ | 6380 | 88% |
| plus MgSO₄•7H₂O | MgSO₄•7H₂O | | |

TABLE 1b
SOD: SEQ ID NO 6

| | | Stability |
|---|---|---|
| Granule | Formulation | (activity %) |
| Raw granule | Cellulose/Dextrin/Kaolin/Na₂SO₄ | 84% |
| Palm oil coating | Cellulose/Dextrin/Kaolin/Na₂SO₄ | 91% |
| (30 wt %) | | |
| Salt coating | Cellulose/Dextrin/Kaolin/Na₂SO₄ | 83% |
| (33 wt %) | | |
| Raw granule plus | Cellulose/Dextrin/Kaolin/Na₂SO₄/ | 91% |
| MgSO₄•7H₂O | MgSO₄•7H₂O | |
| Palm oil coating | Cellulose/Dextrin/Kaolin/Na₂SO₄/ | 90% |
| plus MgSO₄•7H₂O | MgSO₄•7H₂O | |
| Salt coating | Celllose/Dextrin/Kaolin/Na₂SO₄/ | 88% |
| plus MgSO₄•7H₂O | MgSO₄•7H₂O | |

Conclusions and Observation

The fungal SODs of SEQ ID NO: 2 and SEQ ID NO: 6 are highly stable under thermal stress conditions of pelleting. A wide variety of fungal SODs (data not shown) are remarkably stable to thermal stress under a variety of formulations including the raw granule.

The addition of MgSO₄·7H₂O provides an improved stability in all granules.

### Example 8

The thermal stress conditions of Example were duplicated on the catalase of SEQ ID NO:6.

From the result it can be concluded that the presence of SOD stabilize the catalase during the heat and humidity treatment. The catalase used is the polypeptide from Thermoascus aurantiacus having catalase activity sold under the tradename Terminox™ (SEQ ID NO:6).

The Example illustrates that even SEQ ID NO 3, one of the lower performing SODs within the invention, provides the stabilizing effect on the catalase. The example was performed with the SODs of the invention and demonstrated high stabilizing effect on catalases

| | | CAT | SOD |
|---|---|---|---|
| | | Stability | Stability |
| Granule | Formulation | (activity %) | (activity %) |
| Catalase of SEQ ID | Cellulose/Dextrin/ | 2% | — |
| NO: 6 | Kaolin/Na₂SO₄ | | |
| SOD of SEQ ID NO:3 | Cellulose/Dextrin/ | — | (16%) |
| | Kaolin/Na₂SO₄ | | |
| SEQ ID NO: 6 + | Cellulose/Dextrin/ | 63% | (22%) |
| SEQ ID NO: 3 | Kaolin/Na₂SO₄ | | |

1. (canceled)

2. (canceled)

3. A granule comprising a polypeptide having superoxide dismutase activity and a polypeptide having catalase activity, wherein the molar ratio between the polypeptide having superoxide dismutase activity and the polypeptide having catalase activity is greater than 5:1.

4. (canceled)

5. The granule of claim 3, wherein the granule is a coated granule comprising a core and a coating layer surrounding the core.

6. The granule claim 5, wherein the polypeptide having superoxide dismutase activity and the polypeptide having catalase activity are present in the core of the granule.

7. The granule of claim 3, wherein the amount of polypeptide having superoxide dismutase activity is between 1-10% w/w of the granule.

8. The granule claim 3, wherein the amount of polypeptide having catalase activity is between 0.02-1.5% w/w of the granule.

9. The granule of claim 3, wherein the granule has a particle size which is between 50 μm-2000 μm.

10. The granule of claim 5, wherein the core comprises an inert particle.

11. The granule of claim 5, wherein the core comprises one or more ingredients selected from the group consisting of: salts, carriers, particulate fillers and granulation binders.

12. The granule of claim 11, wherein the core comprises one of or more salts selected from the group consisting of: Na₂SO₄, K₂SO₄, KHSO₄, ZnSO₄, MgSO₄, CuSO₄, (NH₄)₂SO₄, Mg(CH₃COO)₂, Zn(CH₃COO)₂, magnesium acetate, and sodium citrate.

13. The granule of claim 5, wherein the coating layer surrounding the core comprises one or more of oils and/or waxes selected from the group consisting of: castor oil, hydrogenated castor oil, palm kernel oil, hydrogenated palm kernel oil, palm oil, hydrogenated palm oil, hydrogenated cotton seeds, soy bean oil, hydrogenated soy bean oil, rapeseed oil, hydrogenated rapeseed oil, a blend of hydrogenated and unhydrogenated vegetable oil, 12-hyroxystearic acid, microcrystalline wax, high-melting paraffin waxes and mixtures thereof.

14. The granule of claim 3, wherein the activity of the polypeptide having superoxide dismutase (SOD) activity present in the granule after steam pelleting at 90 degrees Celsius is retained at least 60% compared to the activity of the polypeptide having superoxide dismutase (SOD) activity in the granules before steam pelleting.

15. The granule of claim 3, wherein the activity of the catalase present in the granule after steam pelleting at 90 degrees Celsius is retained at least 60% compared to the activity of the catalase in the core of the granule before steam pelleting.

16. (canceled)

17. (canceled)

18. (canceled)

19. (canceled)

20. The granule of claim 15, wherein the steam pelleting is at 90 degrees Celsius and 95% relative humidity, for a conditioning time of 90 sec.

21. The granule of claim 3, where in the polypeptide having superoxide dismutase activity is selected from the group consisting of SEQ ID NO: 1, SEQ ID NO: 2, SEQ ID NO: 3, SEQ ID NO: 4, SEQ ID NO: 5.

22. The granule of claim 3, wherein the polypeptide having catalase activity is selected from the group consisting of SEQ ID NO: 6, SEQ ID NO: 7, SEQ ID NO: 8 and SEQ ID NO: 9.

23. (canceled)

24. (canceled)

25. An animal feed composition comprising the granule of claim 3.

26. An animal feed composition comprising the granule of claim 5.

27. (canceled)

28. (canceled)

29. (canceled)

30. (canceled)

31. (canceled)


SEQUENCE LISTING (11 sequences)

SEQ ID NO: 1 - protein, 287 aa, Trichoderma reesei
KDAPVVIDNPQGVEFKASLPKKPFFAGAALVGNVKGSVSATAGPAGKGVRFRVQFENLPKTGGPFLYHIHEEPAVNGNCTSTLAHLDPYHRGETPACNASEPQTCQVGDLSGKYGKITSDPFVAEYFDLYTSLQPGSPAFFGNRSIVIHYANKTRLTCANFAKLPVPAATTVPSTTGAHHTAIHGPLATGGAQHNLTGSHNTTGHHNFTSPHRTTGAASPSDVYTLTSVVTVPGSPTTSSSLTTTDDAGSGGASATSSVVPFPGAASGNRVSLSLLSGVVAAIFFSL

SEQ ID NO: 2 - protein, 162 aa, Aspergillus japonicus
DTSAPIVWDNNDSVWQVQLLHKDNTTVQGHLKASPGPDNVGITIHAKFWGIPTDQQPLLYHIHKKRVPEDGNCYSTSSHLDPYGRTDTPPCDIKAPQTCEVGDLSGKHGPIWAAPGEPVAVSYTDWFLSNVEDTPGFIGNLSVVVHAADNTRLTCGNFEEAD

SEQ ID NO: 3 - protein, 166 aa, Aspergillus templicola
QDKTAPVVTDNKPWRTYTAELQDKDNTTLRGQVTTYGPTDGVGIRLKVSFWGIPEGETLQYHIHAKPVPEDGNCYATAKHLDPYGRTDTPPCDIKNPASCEVGDLSGKHGPVWVPSGHSFNAEYTDYFLSNVEGEEAFFGDLSVVVHASDTSRLNCGNFKGFFESD

SEQ ID NO: 4 - protein, 192 aa, Diaporthe nobilis
QDTGKLGDAAVINDNPVGAVYKATLPETAFTKSAYPNGGNVKGSVYALSSPDGTGVIFKVKFSGLPSEGGPFIYHLHDQPVPEDGNCTSTLAHLDPYERGETTPCDPNAPQTCQVGDLSGKHGNITGDTDATYTDNFASLKEGIGAFFGNRSLVFHFGNKTRITCANFEAVNAGNTCSGSPSSTASSTGTSA

SEQ ID NO: 5 - protein, 159 aa, Armillaria ostoyae
DSLVSQAVSVMTGPGQVKGTVIFQQSSVDGPVHITGRIEGLDPSALRGFHVHTSGNLTGGCDSTGEHWNPFNTNHGGLQDPKDQRHVGDLGNVKTDANGVATLDITDDIMSLNGPLSIVGRAIVVHAGTDDLGKGGTPLSLVNGNSGARVSCGVIVSTF

SEQ ID NO: 6 - protein, 740 aa, Thermoascus aurantiacus
MRAIGLLPGIIGIAGAACPYMTGELPRSFAENPHAINRRAEGGGGAAAETEKFLSQFYLNDNDTFMTTDVGGPIEDQNSLSAGDRGPTLLEDFILRQKIQRFDHERVPERAVHARGAGAHGVFTSYADWSNITAASFLSAAGKETPVFVRFSTVAGSRGSADTARDVHGFATRFYTDEGNFDIVGNNIPVFFIQDAIQFPDLIHAVKPSPNNEIPQAATAHDSAWDFFSQQPSSLHTLFWAMAGHGIPRSYRNMDGFGIHTFRFVTDDGASKLVKFHWTSLQGKASLVWEEAQAVAGKNADYHRQDLWDAIEAGRYPEWELGVQIMDEEDQLRFGFDLLDPTKIVPEEYVPITKLGKMQLNRNPLNYFAETEQIMFQPGHVVRGIDFTEDPLLQGRLFSYLDTQLNRHGGPNFEQIPINRPRTPIHNNNRDGAAQMYIPLNKAAYTPNTLNNGSPKQANQTVGKGFFTTPGRTASGRLVRAVSSTFADVWSQPRLFYNSLVPAEQQFLINAIRFETAHITSDVVKNNVIIQLNRVSNNLAKRVARAIGVAEPEPDPTLYHNNKTANVGVFGKPLARLDGLQVGVLATVNKPDSIKQAASLKASFAADNVDVKVVAERLADGVDETYSAADAVNFDAILVANGAEGLFARDSFTARPANSTTATLYPAGRPLQILVDGFRYGKPVGALGSGAKALDAAEISTTRAGVYVANSTTDSFINGVRDGLRTFKFLDRFAIDEDAE

SEQ ID NO: 7 - protein, 729 aa, Thermoascus aurantiacus
ACPYMTGELPRSFAENPHAINRRAEGGGGAAAETEKFLSQFYLNDNDTFMTTDVGGPIEDQNSLSAGDRGPTLLEDFILRQKIQRFDHERVPERAVHARGAGAHGVFTSYADWSNITAASFLSAAGKETPVFVRFSTVAGSRGSADTARDVHGFATRFYTDEGNFDIVGNNIPVFFIQDAIQFPDLIHAVKPSPNNEIPQAATAHDSAWDFFSQQPSSLHTLFWAMAGHGIPRSYRNMAGFGIHTFRFVTDDGASKLVKFHWTSLQGKASLVWEEAQAVAGKNADYHRQDLWDAIEAGRYPEWELGVQIMDEEDQLRFGFDLLDPTKIVPEEYVPITKLGKMQLNRNPLNYFAETEQIMFQPGHVVRGIDFTEDPLLQGRLFSYLDTQLNRHGGPNFEQIPINRPRTPIHNNNRDGAAQMYIPLNKAAYTPNTLNNGSPKQANQTVGKGFFTTPGRTASGRLVRAVSSTFADVWSQPRLFYNSLVPAEQQFLINAIRFETAHITSDVVKNNVIIQLNRVSNNLAKRVARAIGVAEPEPDPTLYHNNKTANVGVFGKPLARLDGLQVGVLATVNKPDSIKQAASLKASFAADNVDVKVVAERLADGVDETYSAADAVNFDAILVANGAEGLFARDSFTARPANSTTATLYPAGRPLQILVDGFRYGKPVGALGSGAKALDAAEISTTRAGVYVANSTTDSFINGVRDGLRTFKFLDRFAIDEDAETSHHH

SEQ ID NO: 8 - protein, 729 aa, Thermoascus aurantiacus
ACPYMTGELPRSFAENPHAINRRAEGGGGAAAETEKFLSQFYLNDNDTFMTTDVGGPIEDQNSLSAGDRGPTLLEDFILRQKIQRFDHERVPERAVAARGAGAHGVFTSYADWSNITAASFLSAAGKETPVFVRFSTVAGSRGSADTARDVHGFATRFYTDEGNFDIVGANIPVFFIQDAIQFPDLIHAVKPSPNNEIPQAATAHDSAWDFFSQQPSSLHTLFWAMAGHGIPRSYRNMDGFGIHTFRFVTDDGASKLVKFHWTSLQGKASLVWEEAQAVAGKNADYHRQDLWDAIEAGRYPEWELGVQIMDEEDQLRFGFDLLDPTKIVPEEYVPITKLGKMQLNRNPLNYFAETEQIMFQPGHVVRGIDFTEDPLLQGRLFSYLDTQLNRHGGPNFEQIPINRPRTPIHNNNRDGAAQMYIPLNKAAYTPNTLNNGSPKQANQTVGKGFFTTPGRTASGRLVRAVSSTFADVWSQPRLFYNSLVPAEQQFLINAIRFETAHITSDVVKNNVIIQLNRVSNNLAKRVARAIGVAEPEPDPTLYHNNKTANVGVFGKPLARLDGLQVGVLATVNKPDSIKQAASLKASFAADNVDVKVVAERLADGVDETYSAADAVNFDAILVANGAEGLFARDSFTARPANSTTATLYPAGRPLQILVDGFRYGKPVGALGSGAKALDAAEISTTRAGVYVANSTTDSFINGVRDGLRTFKFLDRFAIDEDAETSHHH

SEQ ID NO: 9 - protein, 729 aa, Thermoascus aurantiacus
ACPYMTGELPRSFAENPHAINRRAEGGGGAAAETEKFLSQFYLNDNDTFMTTDVGGPIEDQNSLSAGDRGPTLLEDFILRQKIQRFDHERVPERAVAARGAGAHGVFTSYADWSNITAASFLSAAGKETPVFVRFSTVAGSRGSADTARDVHGFATRFYTDEGNFDIVGNNIPVFFIQDAIQFPDLIHAVKPSPNNEIPQAATAHDSAWDFFSQQPSSLHTLFWAMAGHGIPRSYRNMDGFGIHTFRFVTDDGASKLVKFHWTSLQGKASLVWEEAQAVAGKNADYHRQDLWDAIEAGRYPEWELGVQIMDEEDQLRFGFDLLDPTKIVPEEYVPITKLGKMQLNRNPLNYFAETEQIMFQPGHVVRGIDFTEDPLLQGRLFSYLDTQLNRHGGPNFEQIPINRPRTPIHNNNRDGAAQMYIPLNKAAYTPNTLNNGSPKQANQTVGKGFFTTPGRTASGRLVRAVSSTFADVWSQPRLFYNSLVPAEQQFLINAIRFETAHITSDVVKNNVIIQLNRVSNNLAKRVARAIGVAEPEPDPTLYHNNKTANVGVFGKPLARLDGLQVGVLATVNKPDSIKQAASLKASFAADNVDVKVVAERLADGVDETYSAADAVNFDAILVANGAEGLFARDSFTARPANSTTATLYPAGRPLQILVDGFRYGKPVGALGSGAKALDAAEISTTRAGVYVANSTTDSFINGVRDGLRTFKFLDRFAIDEDAETSHHH

SEQ ID NO: 10 - protein, 729 aa, Thermoascus aurantiacus
ACPYMTGELPRSFAENPHAINRRAEGGGGAAAETEKFLSQFYLNDNDTFMTTDVGGPIEDQNSLSAGDRGPTLLEDFILRQKIQRFDHERVPERAVHARGAGAHGVFTSYADWSNITAASFLSAAGKETPVFVRFSTVAGSRGSADTARDVHGFATRFYTDEGNFDIVGANIPVFFIQDAIQFPDLIHAVKPSPNNEIPQAATAHDSAWDFFSQQPSSLHTLFWAMAGHGIPRSYRNMDGFGIHTFRFVTDDGASKLVKFHWTSLQGKASLVWEEAQAVAGKNADYHRQDLWDAIEAGRYPEWELGVQIMDEEDQLRFGFDLLDPTKIVPEEYVPITKLGKMQLNRNPLNYFAETEQIMFQPGHVVRGIDFTEDPLLQGRLFSYLDTQLNRHGGPNFEQIPINRPRTPIHNNNRDGAAQMYIPLNKAAYTPNTLNNGSPKQANQTVGKGFFTTPGRTASGRLVRAVSSTFADVWSQPRLFYNSLVPAEQQFLINAIRFETAHITSDVVKNNVIIQLNRVSNNLAKRVARAIGVAEPEPDPTLYHNNKTANVGVFGKPLARLDGLQVGVLATVNKPDSIKQAASLKASFAADNVDVKVVAERLADGVDETYSAADAVNFDAILVANGAEGLFARDSFTARPANSTTATLYPAGRPLQILVDGFRYGKPVGALGSGAKALDAAEISTTRAGVYVANSTTDSFINGVRDGLRTFKFLDRFAIDEDAETSHHH

SEQ ID NO: 11 - protein, 729 aa, Thermoascus aurantiacus
ACPYMTGELPRSFAENPHAINRRAEGGGGAAAETEKFLSQFYLNDNDTFMTTDVGGPIEDQNSLSAGDRGPTLLEDFILRQKIQRFDHERVPERAVHARGAGAHGVFTSYADWSNITAASFLSAAGKETPVFVRFSTVAGSRGSADTARDVHGFATRFYTDEGNFDIVGNNIPVFFIQDAIQFPDLIHAVKPSPNNEIPQAATAHDSAWDFFSQQPSSLHTLFWAMAGHGIPRSYRNMDGFGIHTFRFVTDDGASKLVKFHWTSLQGKASLVWEEAQAVAGKNADYHRQDLWDAIEAGRYPEWELGVQIMDEEDQLRFGFDLLDPTKIVPEEYVPITKLGKMQLNRNPLNYFAETEQIMFQPGHVVRGIDFTEDPLLQGRLFSYLDTQLNRHGGPNFEQIPINRPRTPIHNNNRDGAAQMYIPLNKAAYTPNTLNNGSPKQANQTVGKGFFTTPGRTASGRLVRAVSSTFADVWSQPRLFYNSLVPAEQQFLINAIRFETAHITSDVVKNNVIIQLNRVSNNLAKRVARAIGVAEPEPDPTLYHNNKTANVGVFGKPLARLDGLQVGVLATVNKPDSIKQAASLKASFAADNVDVKVVAERLADGVDETYSAADAVNFDAILVANGAEGLFARDSFTARPANSTTATLYPAGRPLQILVDGFRYGKPVGALGSGAKALDAAEISTTRAGVYVANSTTDSFINGVRDGLRTFKFLDRFAIDEDAETSHHH